(19) United States
(12) Patent Application Publication
(10) Pub. No.: US 2003/0194700 A1
(43) Pub. Date: Oct. 16, 2003

(54) Biochip and method for producing the same

(76) Inventors: Toshiaki Ito, Kanagawa (JP); Kenji Yamamoto, Kanagawa (JP); Toshimasa Watanabe, Kanagawa (JP); Noriko Yurino, Kanagawa (JP)

Correspondence Address: MI K. KIM, Fish & Richardson P.C., Suite 500, 4350 La Jolla Village Drive, San Diego, CA, 92122, US

(21) Appl. No.: 09/879,797

(22) Filed: Jun. 11, 2001

Related U.S. Application Data
(63) Continuation-in-part of application No. 09/451,666, filed on Nov. 30, 1999.

(30) Foreign Application Priority Data
Dec. 1, 1998 (JP) 341604/1998

(51) Int. Cl. C12Q 1/68; G01D 15/04; G01D 15/20; C12M 1/34

(52) U.S. Cl. 435/6; 435/287.2; 346/106

(57) ABSTRACT

A device for making a biochip comprising probes spotted on a plate at a plurality of positions by using a binding agent for binding the probes to the plate, wherein the binding agent is locally spotted at positions where the probes are spotted.

### FIELD OF THE INVENTION

The present invention relates to a device for making a biochip spotted with various probes.

### BACKGROUND OF THE INVENTION

Conventionally, biochips are produced by spotting various biopolymer probes such as DNAs, RNAs and proteins on a plate (e.g., a glass plate). FIGS. 4A to 4E are diagrams for illustrating the principle of such a conventional technique. First, a microplate 2 containing various probe DNAs 1 (FIG. 4A) and a glass plate 3 (FIG. 4B) is prepared. As shown in FIG. 4C, the surface of the glass plate 3 is coated with poly-1-lysine binding agent 4 for binding the DNAs 1 to the glass plate 3. Thereafter, each of the probe DNAs 1 in the microplate 2 is transferred by a pin 5 and spotted onto the glass plate 3 coated with the poly-1-lysine binding agent 4 (FIG. 4D). This process is repeated for all of the probe DNAs 1 in the microplate 2, thereby producing a biochip as shown in FIG. 4E. In such a manner, the binding agent for binding DNA to the glass plate is conventionally coated on the entire surface of the plate before spotting the DNAs on the plate.

FIGS. 5A to 5C are diagrams for illustrating the principle of hybridization using the biochip. Referring to FIG. 5A, sample DNA 11 labeled with a fluorescent substance 10 is hybridized in a hybridization solution with the probe DNAs 1 that are spotted onto the glass plate 3 of the biochip via the binding agent 4. The hybridization solution contains formaldehyde, SSC (NaCl, trisodium citrate), SDS (sodium dodecyl sulfate), EDTA (ethylenediamidete triacetic acid), distilled water and the like where the mixing ratio depends on the characteristics of the DNA used.

When the sample DNA 11 is complementary to any one of the probe DNAs 1 on the biochip, it binds to that DNA on the biochip and forms a duplex. The sample DNA 11 does not bind to probe DNAs that are not complementary thereto. However, the sample DNA 11 may bind to the binding agent 4 coating the glass plate 3, thereby remaining as garbage.

As shown in FIG. 5B, the glass plate 3 of the biochip hybridized with the sample DNA 11 is washed in water 12 to remove the sample DNA 11 that is not bound to the probe DNAs 1. Referring to FIG. 5C, the fluorescent substance 10 labeling the sample DNA 11 bound to the probe DNA 1 is excited with light from a lamp 14. The fluorescent light emanating from the fluorescent substance 10 is detected by an optical sensor 13 such as a CCD to detect for the presence of hybridization.

In a laboratory, the sample DNA 11 is poured onto the biochip to allow hybridization with the probe DNAs 1 spotted on the biochip followed by detection of the probe DNA bound by the sample DNA 11. Following the hybridization, but prior to the detection, the biochip is washed with water to remove the sample DNA 11 that did not bind to the probe DNAs 1. However, since the entire surface of the glass plate 3 is coated with the binding agent 4 for binding the probe DNA 1 to the glass plate 3, sample DNA 11 adheres to the binding agent area of the glass plate 3 where the probe DNAs 1 are not located. The sample DNA 11 bound to the binding agent 4 cannot be removed from the glass plate 3 by washing with water. Such remainder sample DNA 11 is detected as noise upon detection, rendering the detection sensitivity poor. In other words, some of the sample DNA 11 that is not specific to the probe DNA 1 binds to and remains on the biochip via the binding agent 4 as garbage. When the fluorescent substance 10 labeling the sample DNA 11 bound to the binding agent 4 is excited, the fluorescent light therefrom is detected as noise, whereby the S/N (signal-to-noise) ratio is lowered.

The present invention aims to solve this problem, and provides a biochip in which sample DNA does not bind to areas of the plate where the probes are not located. The present invention also provides a method for producing such a biochip.

### SUMMARY OF THE INVENTION

In order to accomplish the above objectives, the present invention provides a binding agent for binding probes on a plate only where the probes are to be spotted. Since no binding agent is provided on the portions of the plate where the probes are not to be spotted, the sample DNA that does not bind to the probe upon hybridization can be removed away from the biochip by washing with water. Therefore, noise produced upon detection can be eliminated and, thus, the S/N ratio can be enhanced for high sensitivity.

A biochip according to the present invention includes probes spotted on a plate at a plurality of positions by using a binding agent for binding the probes to the plate, wherein the binding agent is locally spotted at positions where the probes are spotted. Accordingly, the reagents of interest are spotted on the plate. The reagents can be a probe and/or a binding agent, or a mixture of the probe and binding agent. The reagents may be spotted by pins. The pin may include a spotting pin of the present invention.

In a preferred embodiment of the inventive biochip, the material of the plate is selected from the group comprising glass, nylon membranes, silicone wafer, polyimide resin and polymer plastic.

In a further preferred embodiment of the inventive biochip, the binding agent is selected from the group comprising poly-l-lysine, carbodimide, silylation-coating and the like.

A method for producing a biochip by spotting probes on a plate by using a binding agent for binding the probes to the plate according to the present invention includes the step of spotting mixtures of respective probes and the binding agent on the plate.

An alternative method for producing a biochip by spotting probes on a plate according to the present invention includes the steps of: spotting a binding agent for binding the probes to the plate at positions where the probes are to be spotted; and spotting the probes on the plate at positions where the binding agent is spotted.

The plate used in the inventive methods may be made of a material selected from the group comprising glass, nylon membranes, silicone wafer, polyimide resin and polymer plastic

In a preferred embodiment of the inventive methods, the binding agent is selected from the group comprising poly-1-lysine, carbodimide and silylation-coating.

The probes are preferably spotted by using the inventive spotting pin.

The problem underlying the present invention is also solved by a pin used for spotting a probe on a plate, wherein a tip of the pin comprises at least a recess

In one embodiment of the inventive spotting pin, the recess is of a concave shape.

In a second embodiment of the inventive spotting pin, the recess comprises at least one groove.

In a third embodiment, the recess comprises a radially-shaped groove.

According to the invention, a pin used for spotting a probe on a plate has a tip provided with at least one groove. For example, the groove may be a radially-shaped groove such as a cross-shaped groove.

This specification includes all or part of the contents as disclosed in the specification and/or drawings of Japanese Patent Application No. 10-341604, which is a priority document of the present application.

### BRIEF DESCRIPTION OF THE DRAWINGS

FIGS. 1A to 1E are schematic diagrams showing the principle of one embodiment of the present invention;

FIGS. 2A to 2E are diagrams showing the principle of another embodiment of the present invention;

FIGS. 3A to 3C are diagrams for illustrating the principle of hybridization and detection using the biochip of the invention;

FIGS. 4A to 4E are diagrams for illustrating the principle of a method for producing a conventional biochip;

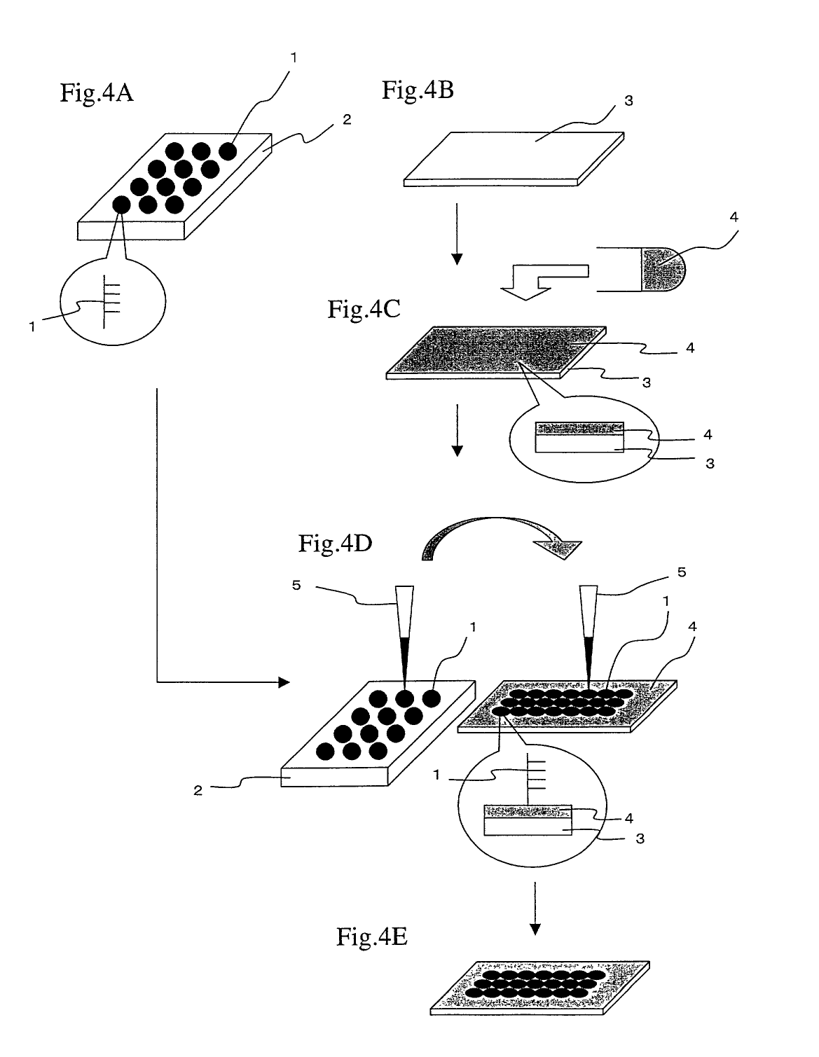

FIGS. 5A to 5C are diagrams for illustrating the principle of hybridization and detection using the conventional biochip; and

FIGS. 6A to 6D are schematic diagrams showing exemplary shapes of a tip (i.e., a portion where probes, binding agent, or probe binding agent mixtures are to be contacted and carried) of a pin according to the invention.

### DETAILED DESCRIPTION OF THE INVENTION

Hereinafter, the present invention will be described in more detail by way of examples with reference to the accompanying drawings. In the examples, DNA is used as a probe although the probe is not limited thereto, and RNA or protein may also be used as a probe. Although a glass plate is used in the examples, a nylon membrane or the like may also be used.

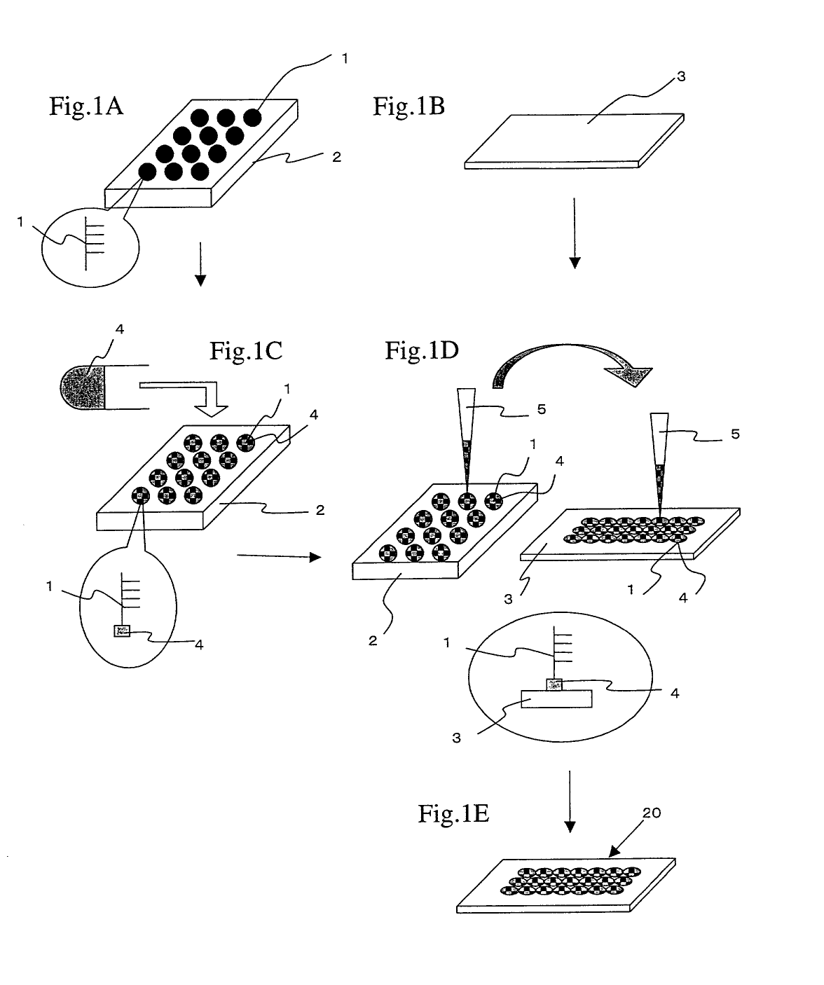

FIGS. 1A to 1E are schematic diagrams showing the principle of a first embodiment of the present invention. As shown in FIG. 1A, a microplate 2 contains various probe DNAs 1. A plate 3 to be incorporated into the biochip shown in FIG. 1B is made of glass. Referring to FIG. 1C, a binding agent 4 for binding DNA to glass is dispensed into each well of the microplate 2 to be mixed therein with each of the probe DNAs 1. The binding agent 4 may be, for example, poly-1-lysine or carbodiimide.

Then, as shown in FIG. 1D, each of the mixtures of the binding agent 4 and the probe DNAs 1 is suctioned by a pin 5 (or contacted and carried by the tip of the pin 5) and spotted onto the plate 3. This process is repeated for all of the probe DNAs 1 in the microplate 2, thereby producing a biochip 20 shown in FIG. 1E in which the binding agent 4 is present only at the desired portions and is not present at portions where there is no probe.

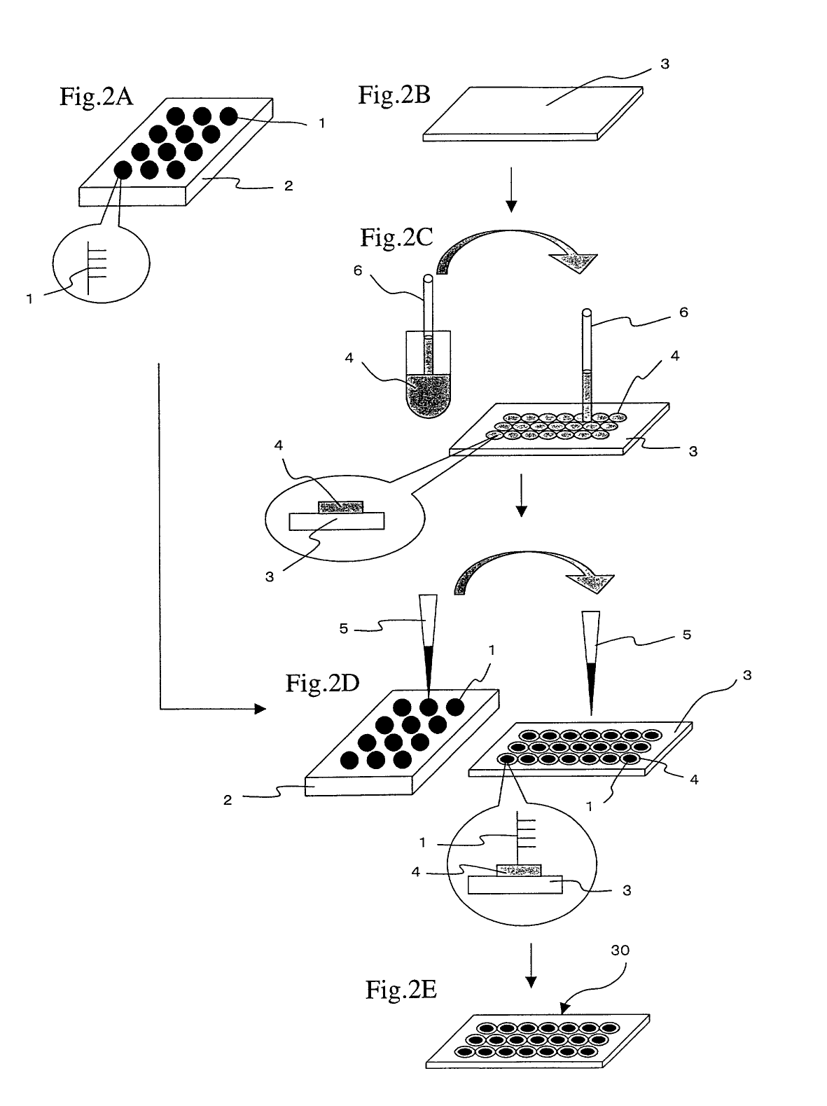

FIGS. 2A to 2E are diagrams showing the principle of a second embodiment of the present invention. A microplate 2 containing various probe DNAs 1 (FIG. 2A) and a plate 3 made of glass (FIG. 2B) is prepared. As shown in FIG. 2C, a binding agent is suctioned by, for example, a pin or a capillary tube 6 and applied on the glass plate 3 at positions where the probe DNAs are to be spotted. Then, as shown in FIG. 2D, the probe DNAs 1 in the microplate 2 are suctioned with the pin 5 (or is contacted and carried by the tip of the pin 5) and spotted onto the plate 3. This process is repeated for all of the probe DNAs 1 in the microplate 2, thereby producing a biochip 30 in which the binding agent 4 is not provided on portions other than portions where the probe DNAs 1 are present (FIG. 2E).

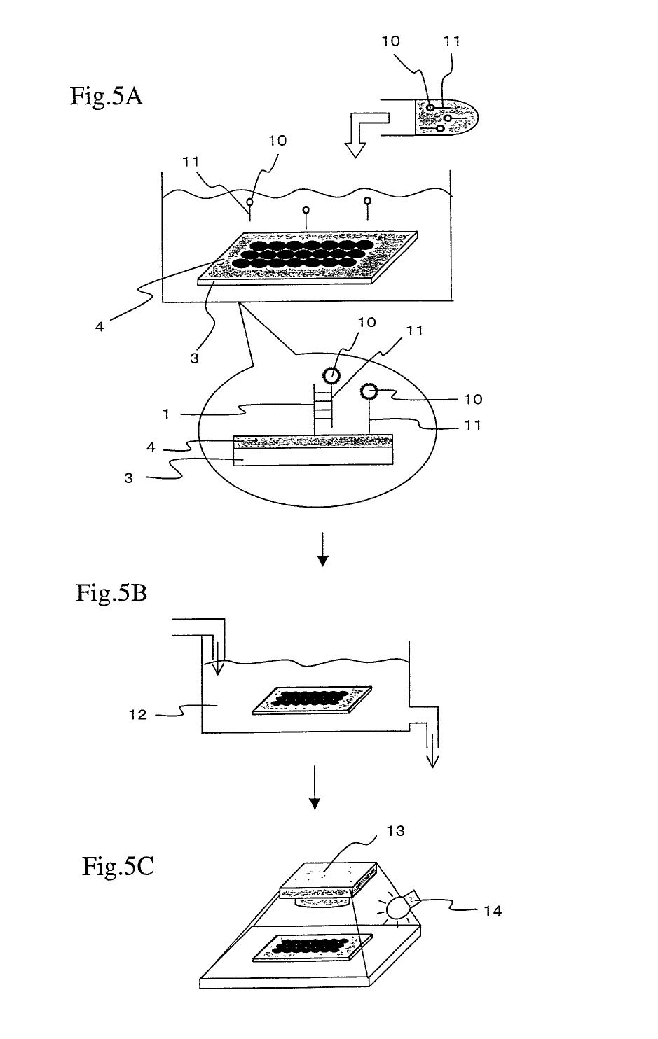

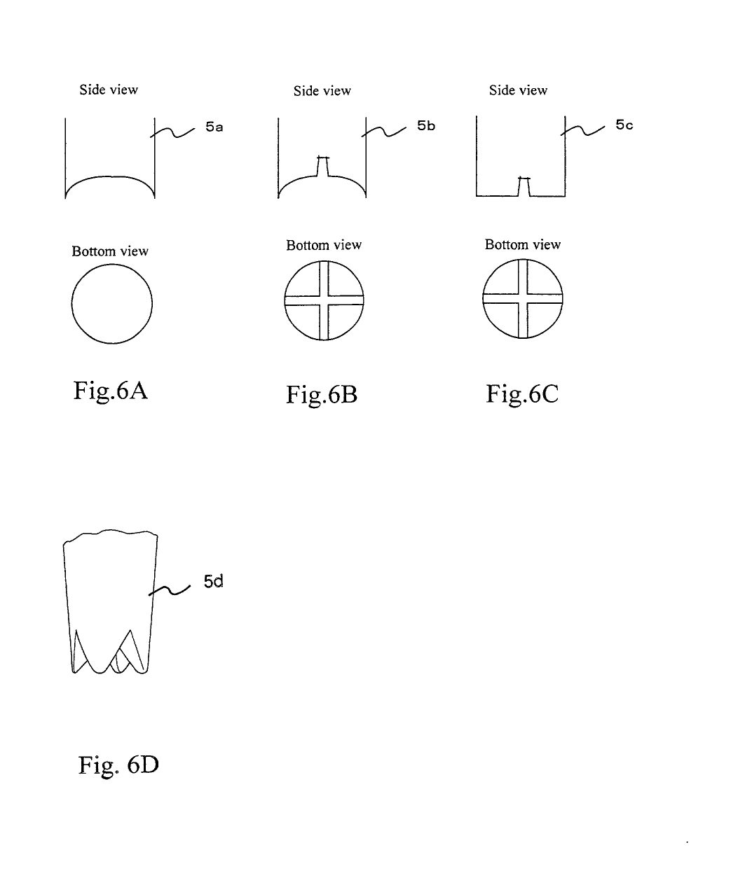

FIGS. 6A to 6D are schematic diagrams showing shapes of a tip (i.e., a portion where probes, binding agent, or a combination thereof are to be contacted) of a spotting pin 5 according to the invention. The spotting pin 5 of the present invention is solid in construct being made of a single material or multiple materials, such as, but not limited to, plastic, metal, metallic alloys or any combination thereof. FIG. 6A shows a pin 5a with a concave tip. A pin 5b shown in FIG. 6B has a concave tip with a cross-shaped groove. The concave shape of the tip of the pin 5b allows the probe solution to be carried by surface tension by simply dipping the pin 5b in the solution. The depth of the concave shape, or the depth of the recess in general, is optional. The amount of DNA carried with the pin 5a or 5b with the concave tip is about 10 times or more the amount carried with a conventional pin with a flat tip. A pin 5c shown in FIG. 5C has a flat tip with a cross-shaped groove. The amount of DNA carried with this pin 5c is also higher than that carried with the conventional flat tip. The pin can also have a V-shaped notch at the recessed tip. The pin 5d shown in FIG. 6D has two V-shaped notches crossing at right angles at its cylindrical head end. With this pin 5d, a greater amount of probe solution, binding agent, or mixture of probe and binding agent can be picked up and spotting accuracy may be enhanced. This pin 5d also allows easy transferring of the probe solution from the tip of the pin 5d onto a plate. The pin may also include a multitude of grooves and/or V-shaped notches (i.e., greater than 2) at the pin's head for enhancing accuracy for spot shape or solution amount control.

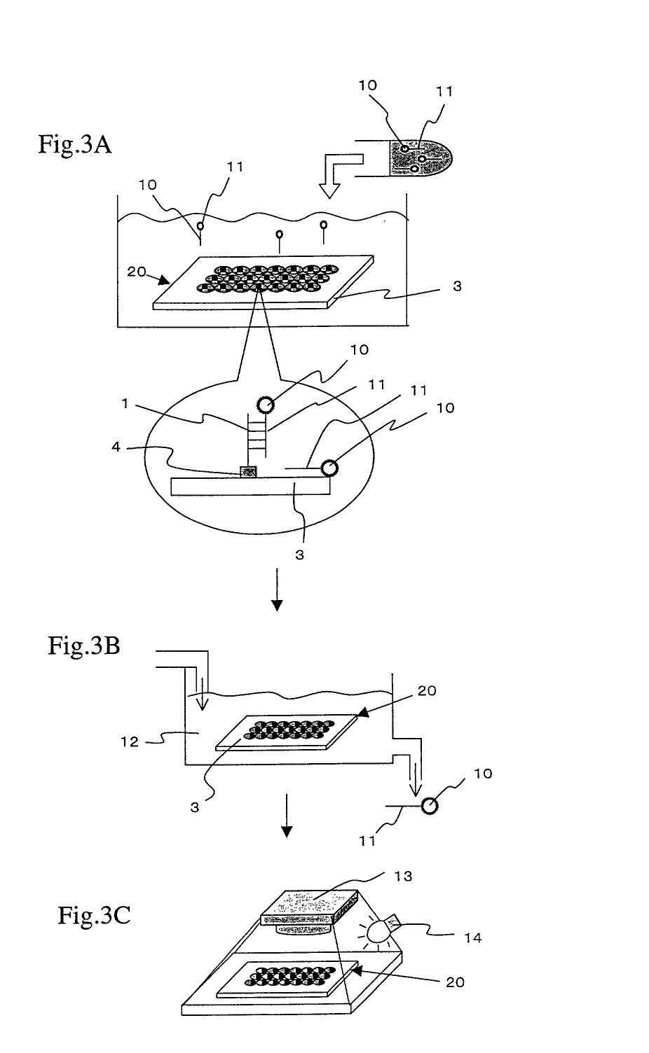

FIGS. 3A to 3C are diagrams for illustrating the principle of hybridization using the biochip 20 of the invention. Referring to FIG. 3A, a sample DNA 11 labeled with a fluorescent substance 10 is placed together with the biochip 20 in a hybridization solution for hybridization. The probe DNAs 1 are spotted on the glass plate 3 via the binding agent 4 in the biochip 20. The hybridization solution contains formaldehyde, SSC (NaCl, trisodium citrate), SDS (sodium dodecyl sulfate), EDTA (ethylenediamidetetraacetic acid) and distilled water, where the mixing ratio differs depending on the characteristic of the DNA used.

When the sample DNA 11 and any one of the probe DNAs 1 on the biochip 20 are complementary to each other, both DNAs bind to each other and form a duplex. On the other hand, when the sample DNA 11 and any one of the probe DNAs 1 are not complementary to each other, the sample DNA 11 does not bind to that probe DNA 1 and remain as garbage. As shown in FIG. 3B, the sample DNA 11 labeled with the fluorescent substance 10 remaining on the glass plate 3 is washed away in water 12. Since the binding between the glass 3 and the DNA 11 is weak, the remaining garbage sample 11 that is not bound to the probe DNAs 1 is removed away. Referring to FIG. 3C, the fluorescent substance 10 labeling the sample DNA 11 bound to the probe DNA 1 is excited with light from a lamp 14. The fluorescent light emanating from the fluorescent substance 10 is detected by an optical sensor 13 such as a CCD to detect the presence of hybridization. Since there is no garbage sample DNA left on the biochip 20, the S/N ratio upon detection is enhanced.

According to the present invention, a biochip can be produced in which a binding agent is locally spotted only where probes are to be spotted. Thus, the detection sensitivity upon reading the biochip can be enhanced.

All publications, including patent and patent application cited herein, are incorporated herein by reference in their entirety.

The features disclosed in the foregoing description, in the claims and/or in the accompanying drawings may, both separately and in any combination thereof, be material for realizing the invention in diverse forms thereof.

What is claimed is:

1. A spotting pin used for spotting a reagent on a plate, wherein a tip of the pin comprises at least one recess.

2. The spotting pin of claim 1, wherein the reagent is any one of a probe, binding agent, or any combination thereof.

3. The spotting pin of claim 1, wherein the recess is of a concave shape.

4. The spotting pin of claim 1, wherein the recess comprises at least one groove.

5. The spotting pin of claim 1, wherein the recess comprises a radially-shaped groove.

6. The spotting pin of claim 1, wherein the recess comprises at least one V-shaped notch.

7. A device for producing a biochip comprising probes spotted on a plate, said device comprising a spotting pin having a tip wherein the tip comprises at least one recess.
